(19) United States
(12) Patent Application Publication
(10) Pub. No.: US 2021/0383929 A1
(43) Pub. Date: Dec. 9, 2021

(54) Systems and Methods for Generating Early Health-Based Alerts from Continuously Detected Data

(71) Applicant: Syntiant, Irvine, CA (US)

(72) Inventor: Pieter Vorenkamp, Laguna Beach, CA (US)

(21) Appl. No.: 17/337,814

(22) Filed: Jun. 3, 2021

Related U.S. Application Data
(60) Provisional application No. 63/124,306, filed on Dec. 11, 2020.
(60) Provisional application No. 63/034,811, filed on Jun. 4, 2020.

(51) Int. Cl. G16H 50/30; G16H 40/67; G16H 10/60

(52) U.S. Cl. CPC G16H 50/30; G16H 10/60; G16H 40/67

(57) ABSTRACT

A voice-based health detection system used to monitor vocal changes of a user to detect early signs of potential health issues. The system may comprise a voice-based health detection server communicatively coupled to sensors such as wearable computing devices. The sensors may be used to capture signal data and monitor vital signs from a user. The server may be configured to receive the signal data from the sensors, identify characteristics from the signal data, and extract features from the characteristics. The extracted features may comprise vocal characteristics of the user such as vocal pitch, speed, range, weight, and timbre. The server may be configured to detect whether the extracted features exceed a predetermined threshold. The system may therefore provide early health-based alerts of potential health issues of the user in response to the detected features exceeding the predetermined threshold and shifting beyond their regular control limits.

### PRIORITY

This application claims the benefit of and priority to U.S. Provisional Application No. 63/124,306, filed Dec. 11, 2020, and U.S. Provisional Application No. 63/034,811, filed Jun. 4, 2020, both of which are incorporated in their entireties herein.

### FIELD

The field of the present disclosure generally relates to artificial intelligence data processing. More particularly, the field of the disclosure relates to processing continuously detected data to generate early-warning health alerts in response to detected changes to one or more known features of a user.

### BACKGROUND

Early detection has been identified as a key factor in the treatment of severe health issues. It is also well-known that there are many early signs for some severe health issues. For example, some of the early signs of a stroke that have been recognized are face drooping, arm weakness, and speech difficulty. Another example are the well-recognized early signs of Alzheimer which may include the increase of body temperature, heart rate, oxygenation saturation (SpO2), and voice pitch amongst others. Some of these early signs can be monitored through continuous tracking of basic vital signs such as blood pressure, heart rate, body temperature, and SpO2.

Currently, many electronic watches and fitness trackers have the capability to continuously monitor and measure various data including some, if not all, of the above-mentioned basic vital signs. And with every new generation of electronic watch and fitness tracker released, monitored data and the analysis of the monitored data by these devices may be used to turn out an increasing amount of meaningful data and correlations. However, most vital signs aside from, for example, body temperature and heart rate typically require users to obtain measurements in a hospital environment, even if many users would much rather stay in the privacy and safety of their own homes. Meanwhile, the few vital signs that may be measured by wearable devices are not currently utilized as these devices have not yet been medically and administratively approved, which implies the accuracy of these devices might not yet meet the minimum medical standards. However, as sensor technology and algorithms continue to drastically evolve, it is therefore very likely that more accurate and reliable data will be produced by these devices in upcoming generation releases.

### BRIEF DESCRIPTION OF THE DRAWINGS

The above, and other, aspects, features, and advantages of several embodiments of the present disclosure will be more apparent from the following description as presented in conjunction with the following several figures of the drawings. The drawings refer to embodiments of the present disclosure in which:

FIG. 1 is an exemplary illustration of a voice-based health detection system, in accordance with an embodiment of the present disclosure;

FIG. 2A is an abstract illustration of a voice-based health detection device, in accordance with an embodiment of the present disclosure;

FIG. 2B is an abstract illustration of known user data, in accordance with an embodiment of the present disclosure;

FIG. 3 is a detailed block diagram illustration of a voice-based health detection server utilized in a voice-based health detection system, in accordance with an embodiment of the present disclosure;

FIG. 4 is a flowchart of a process for generating known user data, in accordance with an embodiment of the present disclosure;

FIG. 5 is a flowchart of a voice-based health detection process, in accordance with an embodiment of the present disclosure;

FIG. 6A is a flowchart of an always-on voice-based health detection process, in accordance with an embodiment of the present disclosure;

FIG. 6B is a flowchart of an always-on voice-based health detection process utilizing an external computing device, in accordance with an embodiment of the present disclosure.

### DETAILED DESCRIPTION

In light of the problems described above, there is a need to monitor changes of a user to generate early health-based alerts of potential health issues from continuously detected data based on the user's monitored changes. The embodiments described herein provide these generated early health-based alerts with systems and methods that are related to detecting and extracting features from audio data signals, which may be used to provide early detection warnings of potential severe health issues for the user. As described in greater detail below, embodiments may allow the user to input one or more features and vitals baseline data that may correspond to one or more spoken words and/or extracted features.

The embodiments may be configured to continuously detect spoken words in a low-power, always-on (i.e., continuously detected) mode, and extract one or more features from the detected spoken words to establish vital baseline data and threshold data (or trend data) of the extracted features over a pre-determined time period. In embodiments described herein, the spoken words may include, but are not limited to, any variety of words, phrases, audio gestures, audio signals, and so on, which may be associated with one or more users. For example, as audio and any related sensor technologies continue to evolve, the embodiments described herein may be capable of detecting one or more features (or parameters, characteristics, etc.) from the voice and spoken words of a user from any general speech voiced by that user, such that the embodiments may detect, parse or otherwise utilize any desired keywords and/or any spoken words from any speech voiced by that user. In other words, features of a user's voice may be detected, parsed or otherwise utilized without the need for a specific key word or pre-programmed phrase to trigger a device or sensor to begin “listening”.

In several embodiments, the extracted features may include one or more vocal characteristics extracted from the detected keywords of the user. For example, the extracted vocal characteristics may include by way of non-limiting example, vocal pitch, vocal speed, vocal range, vocal weight, vocal timbre, and so on. Meanwhile, in other embodiments, the extracted features may include any other health-based data extracted and/or captured with any type of sensors in conjunction with any extracted vocal characteristics. For example, the extracted health-based data may correlate with one or more early signs of health changes that may respectively correlate with one or more potential severe health issues.

Furthermore, embodiments may be configured to determine whether any of the extracted vocal features have exceed a predetermined threshold. In response to determining that one or more of the extracted vocal features have exceed their predetermined thresholds, the embodiments may be configured to generate and transmit alert data including alert notifications of early sign health alert signals to a personal computing device of the user or the user's caregiver. For example, the early sign health alert signal may be used to provide the user with an early warning alert of a potential severe health issue and to promptly seek further diagnosis of the potential issue.

Before the following embodiments are described in greater detail, it should be understood that any of the embodiments described herein do not limit the scope of the concepts provided herein. It should also be understood that a particular embodiment described herein may have features that may be readily separated from the particular embodiment and optionally combined with or substituted for features of any of several other embodiments described herein.

Regarding the terms used herein, it should be understood that the terms are for the purpose of describing particular embodiments and do not limit the scope of the concepts and/or other embodiments described herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “front,” “back,” “top,” “bottom,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

For example, in certain situations, the term “logic” may be representative of hardware, firmware and/or software that is configured to perform one or more functions. As hardware, logic may include circuitry having data processing or storage functionality. Examples of such circuitry may include, but are not limited or restricted to a microprocessor, one or more processor cores, a programmable gate array, a microcontroller, a controller, an application specific integrated circuit, wireless receiver, transmitter and/or transceiver circuitry, semiconductor memory, or combinatorial logic.

Additionally, as used herein, the term “feature” may include any health-based data and any other sensor related data that may be received, transmitted, captured, processed, and/or extract from any type of sensors, any type of sensor processing devices (e.g., any variety of wearable devices), and so on, where such data may be configured to detect and correlate with any early signs of health changes that may respectively correlate with one or more potential sever health issues. For example, any type of sensor may be communicatively coupled to a sensor output detector logic of a voice-based health detection device, where the sensor output detector logic may be configured to identify one or more characteristics (or features, patterns, etc.) from a signal data received by such sensor, and where the sensor output detector logic may be further configured to detect one or more words from the received signal data, such that the identified characteristics may include at least one or more of the detected words. Similarly, as used herein, the term “vocal feature” may include any vocal characteristics extracted from the voice and words voiced by any users in conjunction with any other desired characteristics, which may be extracted and captured with any type of sensors and sensor processing devices to also detect and correlate with any early signs of health changes that may respectively correlate with one or more potential sever health issues.

The term “machine learning” may include any computing circuits that comprise a digital implementation of a neural network. These circuits may include emulation of a plurality of neural structures and/or operations of a biologically based brain and/or nervous system. Some embodiments of machine learning and/or artificial intelligence circuits may comprise probabilistic computing, which may create algorithmic approaches to dealing with uncertainty, ambiguity, and contradiction in received input data. Machine learning circuits may be composed of very-large-scale integration (VLSI) systems containing electronic analog circuits, digital circuits, mixed-mode analog/digital VLSI, and/or software systems.

The term “process” may include an instance of a computer program (e.g., a collection of instructions, also referred to herein as an application). In one embodiment, the process may be included of one or more threads executing concurrently (e.g., each thread may be executing the same or a different instruction concurrently).

The term “processing” may include executing a binary or script, or launching an application in which an object is processed, wherein launching should be interpreted as placing the application in an open state and, in some implementations, performing simulations of actions typical of human interactions with the application.

The term “object” generally refers to a collection of data, whether in transit (e.g., over a network) or at rest (e.g., stored), often having a logical structure or organization that enables it to be categorized or typed. Herein, the terms “binary file” and “binary” will be used interchangeably.

The term “file” is used in a broad sense to refer to a set or collection of data, information or other content used with a computer program. A file may be accessed, opened, stored, manipulated or otherwise processed as a single entity, object or unit. A file may contain other files and may contain related or unrelated contents or no contents at all. A file may also have a logical format, and/or be part of a file system having a logical structure or organization of plural files. Files may have a name, sometimes called simply the “filename,” and often appended properties or other metadata. There are many types of files, such as data files, text files, program files, and directory files. A file may be generated by a user of a computing device or generated by the computing device. Access and/or operations on a file may be mediated by one or more applications and/or the operating system of a computing device. A filesystem may organize the files of the computing device of a storage device. The filesystem may enable tracking of files and enable access of those files. A filesystem may also enable operations on a file. In some embodiments the operations on the file may include file creation, file modification, file opening, file reading, file writing, file closing, and file deletion.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

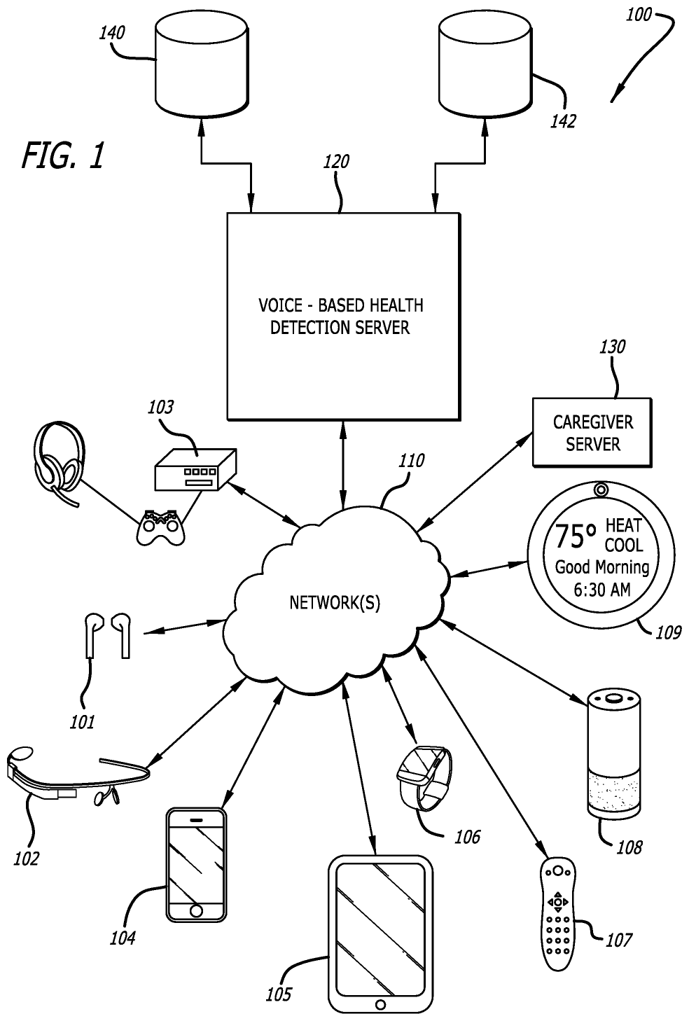

Referring now to FIG. 1, an exemplary illustration of a voice-based health detection system 100 is shown, in accordance with embodiments of the disclosure. In many embodiments, the voice-based health detection system 100 may comprise a plurality of personal computing devices 101-109, a voice-based health detection server 120, a caregiver server 130, and one or more data stores 140 and 142. The voice-based health detection system 100 may utilize and/or otherwise be in communication with the personal computing devices 101-109 that may be configured to monitor for various types of data including, but not limited to, audio data, vital sign data, vocal feature data, and so on of known users to detect early signs of potential health issues of the known users.

As used herein, a known user may be a particular user derived from a variety of sources identified by any of the personal computing devices 101-109. For example, the voice-based health detection system 100 may be configured to particularly identify if a particular keyword is being said by the known user. The known user may be derived from a variety of identified sources which may be included in a predetermined list of authorized known users associated with the particular personal computing device being used. These identified and authorized known users may be associated with a plurality of vocal features within their speech that are unique to that particular known user. These unique features may be utilized to identify particular keywords spoken by the known user against any other words spoken by any unidentified user that may not be associated with the particular computing device and thus not found in the predetermined list of authorized known users.

In many embodiments, the voice-based health detection system 100 may use the personal computing devices 101-109 that may be configured to transmit and receive data related to generating, recording, tracking, and processing known user data, privacy data, threshold data, captured data such as vital signs of the known users, and/or any other signal data, where the voice-based health detection system 100 may respectively generate a plurality of alerts (or alert notifications) based on the transmitted and received data from the sensors in response to one or more vocal features of the known users exceeding a predetermined threshold. In such embodiments, the vocal features may comprise one or more vocal characteristics extracted from the audio data (i.e., extracted vocal features) that are particular to the known user such as, but not limited to, vocal pitch, vocal speed, vocal range, vocal weight, vocal timbre, and/or the like.

As shown in the embodiment depicted in FIG. 1, the voice-based health detection server 120 may be communicatively coupled to one or more network(s) 110 such as, for example, the Internet. The voice-based health detection server 120 may be implemented to transmit a variety of data across the network 110 to any number of computing devices such as, but not limited to, the personal computing devices 101-109, the caregiver server 130, and/or any other computing devices. In additional embodiments, any voice-based health detection data may be mirrored in additional cloud-based service provider servers, edge network systems, and/or the like. In other additional embodiments, the voice-based health detection server 120 may be hosted as one or more virtual servers within a cloud-based service and/or application.

In some embodiments, the transmission of data associated with the voice-based health detection system 100 may be implemented over the network 110 through one or more wired and/or wireless connections. For example, one or more of the personal computing devices 101-109 may be coupled wirelessly to the network 110 via a wireless network access point and/or any wireless devices. As depicted in FIG. 1, the personal computing devices 101-109 may be any type of computing devices capable of capturing audio data and being used by any of the known users, including, but not limited to, a pair of smart hearables 101 such as earbuds, headphones, etc., a head mounted display 102 such as virtual reality head mounted displays, etc., a gaming console 103, a mobile computing device 104, a computing tablet 105, a wearable computing device 106 such as smart watches, fitness watches, etc., a smart remote control 107 such as voice-based tv remote controls, voice-based garage remote controls, voice-based remote control devices/appliances, etc., a smart speaker 108 such as voice-based intelligent personal assistants, voice-based speakers, etc., and a smart home device 109 such as voice-based thermostat controls, voice-based security monitor devices, smart home appliances, voice-based lighting control devices, etc.

In additional embodiments, the personal computing devices 101-109 may be any type of voice-based computing devices. For example, the voice-based computing devices may include any type of portable handheld devices such as a mobile device, a cellular telephone, a mobile or cellular pad, a computing tablet, a personal digital assistant (PDA), any type of wearable devices, any other desired voice-based enabled devices, and/or any of one or more widely-used running software and/or mobile operating systems. The voice-based computing devices may be personal computers and/or laptop computers running various operating systems. The voice-based computing devices may be workstation computers running any variety of commercially available operating systems. Alternatively, the voice-based computing devices may be any other electronic device, such as a thin-client computer, an Internet-enabled gaming system with a messaging input device, and/or a personal voice-enabled messaging device that is capable of communicating over the network 110. Although nine personal computing devices 101-109 are depicted in FIG. 1, it should be understood that any number of computing devices and any types of computing devices may be utilized by the voice-based health detection system 100, without limitation. Also, it should be understood that any types of wired and/or wireless connections between any of the components in the voice-based health detection system 100 may be utilized based on any desired combination of devices, connections, and so on, without limitations.

In various embodiments, the voice-based health detection system 100 may be implemented to continuously receive and monitor voice-based health detection system data, such as, but not limited to, known user data, privacy data, threshold data, captured data, and/or any other signal data, from the known users via any number of personal computing devices 101-109, personal computers, personal listening computing devices, and/or personal mobile computing devices. In many embodiments, the voice-based health detection system data may process a plurality of data related to keywords, vital signs, vitals baseline measurements, and vocal features of the known users; determine whether processed vocal features exceed predetermined thresholds such as dynamic and/or static predetermined thresholds; and generate and transmit alert notifications to the known users and/or the caregiver server 130 in response to the extracted vocal features exceeding the predetermined threshold. Furthermore, in some embodiments, the alert notifications may be generated from a list of predetermined actions within the voice-based health detection server 120, the caregiver server 130, and/or the personal computing devices 101-109.

In other embodiments, the voice-based health detection system data may also be stripped of personal identifying data, such as personal medical history data, and may be transmitted to the voice-based health detection server 120, the caregiver server 130, the data stores 140, 142, and/or any other cloud-based services for processing and/or storing. The processed and/or stored data may then be transmitted back to the personal computing devices 101-109 for output to the known users. For example, the stripped, processed, and stored data may be transmitted using one or more forms of data transmission such as blockchain-based data transmission, hash-based data transmission, encryption-based data transmission, and/or any other similar protected data transmission techniques. In various embodiments, the caregiver server 130 may be implemented to receive (and/or transmit) data related to the vital signs of the known users and any related alert data of the known users, which includes alert notifications received as early warning alerts of potential severe health issues for the known users. For example, the caregiver server 130 may be any servers, computing devices, and/or systems associated with doctors, nurses, and/or any primary caregivers, which have medical-patient relationships with the known users and are qualified to provide further diagnosis for the potential severe health issues.

Additionally, in some embodiments, the voice-based health detection server 120 may be implemented to run one or more voice-based health detection services or software applications provided by one or more of the components of the voice-based health detection system 100. The voice-based health detection services or software applications may include nonvirtual and virtual health monitoring/detecting environments. For some embodiments, these services may be offered as web-based or cloud services or under a Software as a Service (SaaS) model to the known users of any of the personal computing devices 101-109. The known users of any of the personal computing devices 101-109 may in turn use one or more client/user applications to interact with the voice-based health detection server 120 (and/or the caregiver server 130) and utilize the services provided by such servers.

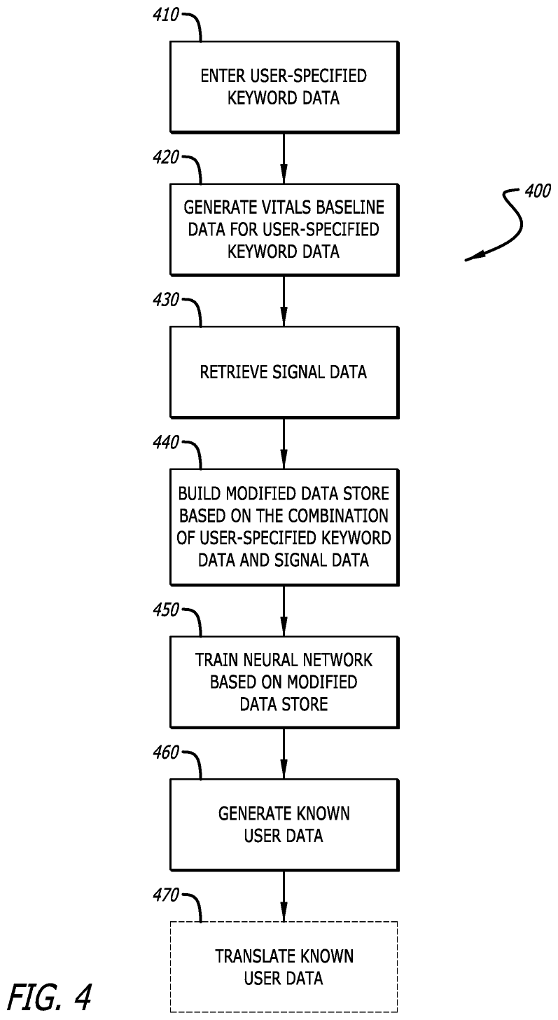

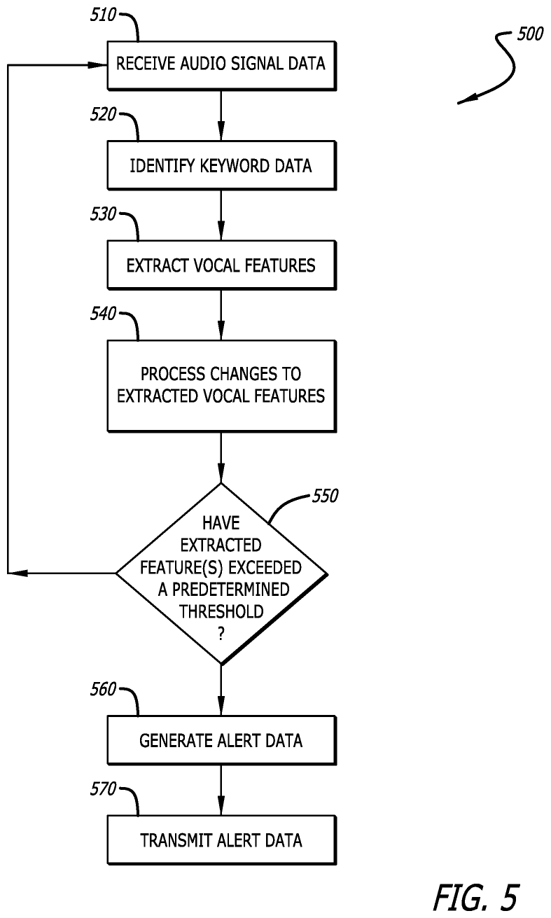

The voice-based health detection server 120 may be configured as personalized computers, specialized server computers (including, by way of non-limiting example, personal computer (PC) servers, mid-range servers, mainframe computers, rack-mounted servers, etc.), server farms, server clusters, and/or any other appropriate desired configurations. The voice-based health detection server 120 may include one or more virtual machines running virtual operating systems, and/or other computing architectures involving virtualization. One or more flexible pools of logical storage devices may be virtualized to maintain virtual storage devices for the voice-based health detection server 120. Virtual networks may be controlled by the voice-based health detection server 120 using software-defined (or cloud-based/defined) networking. In various embodiments, the voice-based health detection server 120 may be configured to run one or more instructions, programs, services, and/or software applications described herein. For example, the voice-based health detection server 120 may be associated with a server implemented to perform any of the processes described below in FIGS. 4, 5, and/or 6A-6B. The voice-based health detection server 120 may implement one or more additional server applications and/or mid-tier applications, including, but are not limited to, hypertext transport protocol (HTTP) servers, file transfer protocol (FTP) servers, common gateway interface (CGI) servers, database servers, and/or the like.

As shown in FIG. 1, the voice-based health detection system 100 may also include the one or more data stores 140 and 142. The data stores 140 and 142 may reside in a variety of locations. By way of non-limiting example, one or more of the data stores 140 and 142 may reside on a non-transitory storage medium local to (and/or resident in) the voice-based health detection server 120. Alternatively, the data stores 140 and 142 may be remote from the voice-based health detection server 120 and in communication with the voice-based health detection server 120 via any desired connections/configurations. In some embodiments, the data stores 140 and 142 may be one or more external medical data stores used to store data related to patient information, private information, and/or medical history of any of the known users. For example, the external medical data stores may be stored remotely from the voice-based health detection server 120 and any of the personal computing devices 101-109.

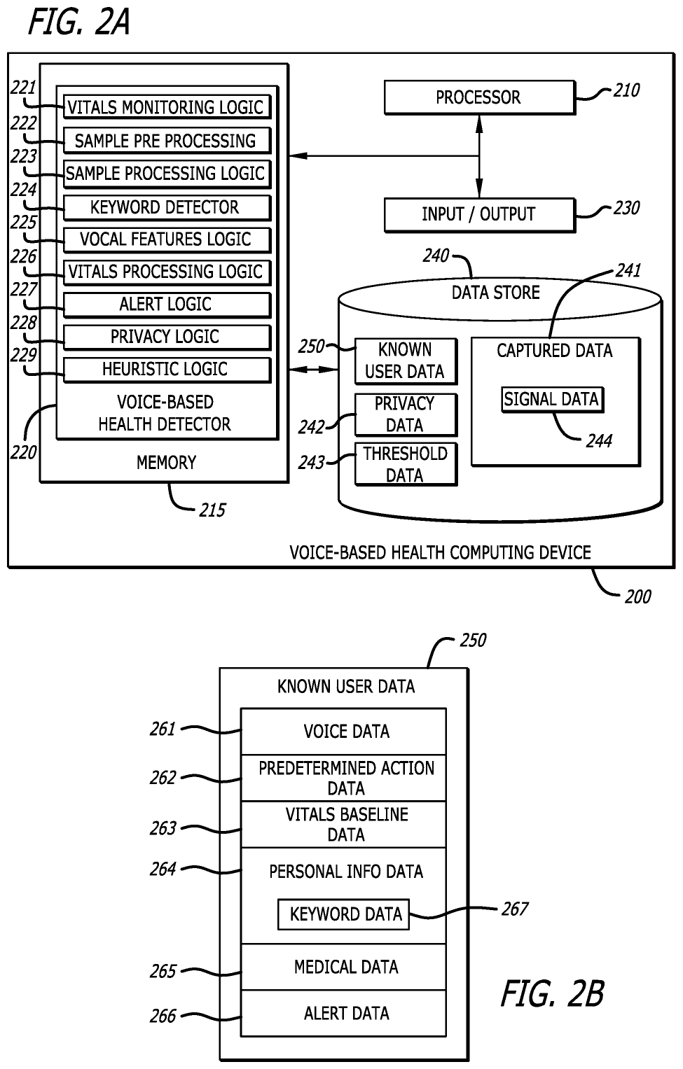

Referring now to FIG. 2A, an abstract illustration of a voice-based health detection device 200 is shown, in accordance with embodiment of the disclosure. In many embodiments, the voice-based health detection device 200 may include a processor 210, a memory 215 with a voice-based health detector application 220, an input/output 230, and a data store 240. The voice-based health detection device 200 depicted in FIG. 2A may be similar to the voice-based health detection server 120 depicted in FIG. 1. For example, the voice-based health detection device 200 may be implemented by the voice-based health detection system 100 in conjunction with any other additional devices, servers, and/or systems such as, but not limited to, one or more of the personal computing devices 101-109 and the caregiver server 130 depicted in FIG. 1. In some embodiments, the voice-based health detection device 200 may be any computing device that may implement a voice-based health detection system process such as the voice-based detection system process 100 depicted in FIG. 1. As noted, the computing devices may include any of the personal computing devices 101-109 of FIG. 1, and/or may comprise any computing device sufficient to receive, transmit, and respond to any voice-based health detection entries from any known users.

In various embodiments, the voice-based health detection device 200 may be communicatively coupled to one of the personal computing devices 101-109 of FIG. 1 which are configured to monitor vocal features and identify changes to the vocal features. Such vocal changes may be used by the voice-based health detection device 200 to identify and detect early signs of potential health issues of known users. In many embodiments, the voice-based health detection device 200 may detect these early health issues by implementing one or more logics within a voice-based health detector application 220 to receive audio data from the sensors, identify keywords from the received audio data, and extract vocal features from the identified keywords.

As illustrated in FIG. 2A, the memory 215 may comprise the voice-based health detector application 220 which may further comprise vitals monitoring logic 221, sample pre-processing logic 222, sample processing logic 223, keyword detector logic 224 (and/or sensor output detector logic), vocal features logic 225, vitals processing logic 226, alert logic 227, privacy logic 228, and/or heuristic logic 229. The data store 240 may include captured data 241, privacy data 242, threshold data 243, signal data 244, and known user data 250.

In a number of embodiments, the vitals monitoring logic 221 may be configured to receive and and/or facilitate transfer of data between the voice-based health detection device 200 and any external computing devices, such as the personal computing devices 101-109 of FIG. 1, external sensor/monitoring services, and so on. For example, the data received by the vitals monitoring logic 221 may be stored as the captured data 241 within the data store 240, where the captured data 241 may include any type of data captured and received by the vitals monitoring logic 221. In some embodiments, the vitals monitoring logic 221 may establish communication channels with the external computing devices via a network connection similar to the network 110 depicted in FIG. 1. Certain embodiments may utilize network connection tools provided by the operating system of the voice-based health detection device 200.

The vitals monitoring logic 221 may be configured to receive signal input from any suitable signal input sources, such as a microphone, an audio data source, and/or a sensor. The microphone may include audio microphones, digital microphones, or other waveform detecting devices. The audio data source may be comprised of any other type of processing data source capable of receiving/detecting/providing various input signals. The sensor may be comprised of any type of sensors and/or sensor-enable devices such as, but not limited to, vital sign monitoring sensors (e.g., sensors used to monitor heart rate, blood pressure, body temperature, oxygen saturation (SpO2), vocal features, etc.), medical sensors, fitness tracking sensors, infrared sensors, pressure sensors, temperature sensors, proximity sensors, motion sensors, fingerprint scanners, photo eye sensors, wireless signal antennae, accelerometers, gyroscopes, magnetometers, tilt sensors, humidity sensors, barometers, light sensors (e.g., ambient light sensors), color sensors, touch sensors, flow sensors, level sensors, ultrasonic sensors, smoke, alcohol, and/or gas sensors (i.e., sensors capable of detecting smoke/alcohol/gas from human airways), and so on. For example, the signal input data received by the vitals monitoring logic 221 via the microphone, audio data source, and/or sensors may be stored as the signal data 244 within the captured data 241 of the data store 240, where the signal data 244 may include any type of signal input data such as audio data, audio signal streams, audio waveform samples, etc.

In many embodiments, the sample pre-processing logic 222 in conjunction with the sample processing logic 223 may be configured to receive, process, and transmit any data related to the captured data 241 with the signal data 244 received by the vitals monitoring logic 221. The sample pre-processing logic 222 may be configured to use the vocal features extracted from the pre-processed sensor data such as the captured data 241 to arrive at one or more actionable decisions by a neural network or the like. In many embodiments, the sample pre-processing logic 222 may be configured as a filter bank or the like that may be used to receive, for example, the captured signal data 244, where the received data of the sample pre-processing logic 222 may be filtered and pre-processed based on the desired actionable decisions prior to feeding such data to the sample processing logic 223. That is, in some embodiments, the sample pre-processing logic 222 may be configured as an enhancement filter or the like that may be configured to suppress undesired noise in a signal by selectively attenuating or boosting certain components of the signal on a time-varying basis, and/or by suppressing undesired noise in a signal by selectively attenuating or boosting certain components of the signal on a time-varying basis. For example, the sample pre-processing logic 222 may be configured as pulse-density modulation (PDM) decimation logic configured to decimate PDM audio samples from any of signal input sources described herein to a baseband audio sampling rate for use in the voice-based health detection device 200.

The sample processing logic 223 may be configured to receive any type of signal data such as frequency elements or signal spectrum information in the form of Fourier transforms or similar frequency decompositions, where the received signal data may processed for audio signal-processing tasks such as audio enhancement, de-noising, and/or the like. In many embodiments, the sample processing logic 223 may receive the audio signal-processing tasks and may be in conjunction with the keyword detector logic 224 that are configured to receive audio input data and subsequently perform word recognition tasks, such as intensifying characteristics from the received input data and so on. For example, as described herein, the keyword detector logic 224 may be a sensor output detector logic configured to identify characteristics, keywords, and such from the received signal data 244 and then the sample processing logic 223 may be configured to respectively generate keyword data (and/or characteristics data, sensor output data, and so on) based on the identified keywords and process the generated keyword data against the known user data 250, as described in further detail below.

In some embodiments, the sample processing logic 223 in conjunction with the keyword detector logic 224 may be utilized to then transmit the identified keywords and generated/processed keyword data to the vocal features logic 225 based on the result(s) aggregated from the performed word recognition tasks of both and/or one of more of the sample processing logic 223 and keyword detector logic 224. In addition, as described in further detail below, the keyword detector logic 224 may have access to one or more data types within the known user data 250 depicted in FIG. 2B, which may include one or more lists of keywords stored within keyword data 267, vocal features stored within vitals baseline data 263, and/or particular voice identification data of the particular known users stored within the voice data 261 and/or personal information data 264.

In various embodiments, the vocal features logic 225 may be configured to extract one or more vocal features from the processed keyword data. For example, the vocal features logic 225 may extract any vocal features associated with vital signs being monitored for the known users, where the extracted vocal features may be stored in the threshold data 243 and the vitals baseline data 263 depicted in FIG. 2B. The vocal features extracted by the vocal features logic 225 may be extracted from any audio signals captured by the vitals monitoring logic 221, where each vocal feature corresponds to one or more particularly monitored vital signs of the known user. The extracted vocal features may comprise vocal characteristics of the known user such as, but not limited to, vocal pitch, speed, range, weight, and timbre. In some embodiments, the vocal features logic 225 may be configured to transfer the extracted vocal features and any processed vitals baseline data to the vitals processing logic 226, which may be configured to detect whether the extracted vocal features exceed their respective predetermined thresholds.

In many embodiments, the vitals processing logic 226 may be configured to process the one or more extracted vocal features against known user vitals data, such as the vitals baseline data 263 depicted in FIG. 2B that is stored in the known user data 250. The vitals processing logic 226 may also be configured to determine whether the one or more processed vocal features exceed one or more predetermined thresholds, such as dynamic and static predetermined thresholds described in greater detail below. In various embodiments, the vitals processing logic may utilize external factors captured by the heuristic logic 229 to facilitate the processing of the extracted vocal features and/or generation of any alert data 266 of FIG. 2B with the alert logic 227 if needed, as described below in greater detail.

In many embodiments, the alert logic 227 may be configured to generate alert data 266 depicted in FIG. 2B in response to the one or more extracted vocal features exceeding the predetermined thresholds stored in the threshold data 243, where the threshold data 243 may be used to determine any trends of the extracted vocal features in relation to the dynamic and/or static predetermined thresholds. The alert logic 227 may also be configured and transmit the generated alert data to one or more computing devices. In many embodiments, the alert logic 227 may be configured to generate and transmit the alert data 266 associated with one or more generated and transmitted alerts of the known users. For example, the alert logic 227 may be configured to generate any known users alerts that were generated and transmitted in response to determining that the extracted features exceeded their respective predetermined thresholds.

In some embodiments, the stored alert data 266 may be generated with the alert logic 227 that may then trigger one or more predetermined actions stored in the predetermined action data 262 depicted in FIG. 2B. The predetermined actions generated by the alert logic 227 may include at least one or more of known user alerts and caregiver alerts based on the triggered predetermined action data 262. The known user alerts may comprise an early warning alert, a warning alert, and an emergency alert. The caregiver alerts may comprise a caregiver early warning alert, a caregiver warning alert, and a caregiver emergency alert. In many embodiments, the known user and caregiver alerts generated by the alert logic 227 may comprise any type of alert notifications used for early health detections of potential severe health issues which are associated with the known users.

In some embodiments, the privacy logic 228 may be configured to receive and transmit any privacy data 242 which may also include any medical history data such as the medical history data 265 depicted in FIG. 2B. The privacy logic 228 may be used for transmitting any privacy data 242 related to any medical information that is private and associated with any of the known users. The privacy logic 228 may be configured to strip any particular privacy data 242 that may not be transmitted and/or may be configured to transmit any privacy data 242 such as the medical history data 265 via blockchain-based data transmission, hash-based data transmission, encryption-based data transmission, and/or any other similar protected data transmission.

For example, in some embodiments, the heuristic logic 229 may be configured to capture a plurality of external factors with the vitals monitoring logic 221 and/or any other monitoring device that may provide supplemental data capable of being used to enhance the determinations of the vitals processing logic 226. In some embodiments, one or more external factors associated with the known user that may be utilized to gain insight into any of the captured data 241 in conjunction with captured voice data 261, vitals baseline data 263, and/or known user data 250 depicted in FIG. 2B. For example, external factors may indicate that a known user has a workout routine during a specified time every week which may naturally cause changes in a user's voice. These determined acute changes can then be utilized to further gain understanding (or at least generate additional data points) when processing changes and/or evaluating thresholds within their voice data 261 and other subsequent data depicted in FIGS. 2A-2B. The external factors may also include any additional data relating to the event and physical location where the data was captured. Some external factors captured with the heuristic logic 229 may include the global positioning system (GPS) coordinates of where the known user lives, where the captured vital sign measurements were taken (e.g., during an outdoor activity, on vacation, at work, etc.), the time at which it was taken (e.g., late at night or first thing in the morning, etc.), what was the quality of the recording, how long the recording was, and so on.

Referring now to FIG. 2B, an abstract illustration of a known user data 250 is shown, in accordance with embodiments of the disclosure. As described above with reference to FIG. 2A, the known user data 250 may exist within the data store 240 and may be unique to each known user that is associated with the device 200. The known user data in FIGS. 2A-2B is depicted as being portioned and stored based on the individual data types associated with the known user. Further discussion of the types of data that may be found within the known user data 250 is depicted below. The known user data 250 may comprise voice data 261, predetermined action data 262, vitals baseline data 263, personal information data 264 with keyword data 267, medical history data 265, and alert data 266. Although six data types 261-266 are shown in FIG. 2B, it should be understood that any number of data types may be utilized and any one or more of the illustrated data types may be omitted, combined, and so on, without limitations. Additionally, it should be understood that the known user data 250 may be utilized for all the known users and/or may also be utilized to store any of the desired data types associated with only one known user, where each of the known users may have their own respective known user data 250 with any number of data types and any types of data store within each of the known user data 250, without limitation.

In many embodiments, the voice data 261 may comprise any voice data that is associated with each particularly known user, which may include differentiating particular vocal features of each known user. For example, the voice data 261 may include voice data of one user that has a speech impairment, while the second user has no issues and the voice data associated with that second user is different than that one user. The voice data 261 may be comprised as raw audio data that is captured with a microphone or other audio recording device during the voice-based health detection process. This voice data 261 may comprise waveform data and can be formatted into any audio format desired based on the application and/or computing resources. For example, limited storage resources may lead to using increased compression algorithms to reduce size, while computing resources may limit the amount of compression that can be done on the fly. The voice data 261 may be stored in lossy or lossless formats. In some embodiments, the voice data 261 may be processed before storage or utilization elsewhere within the voice-based health detection system. Pre-processing can include noise-reduction, frequency equalizing, normalizing, and or compression. Such pre-processing may increase the amount of supplemental data that can be generated from the voice data 261.

In additional embodiments, the predetermined action data 262 may be comprised of one or more actions that are triggered based on the extracted features exceeding their predetermined thresholds. For example, the alert logic 227 of FIG. 2A may be configured to trigger, in response to generated alert data, one or more predetermined actions within the predetermined action data 262. The triggered actions may include at least one or more of known user alerts and caregiver alerts based on the triggered predetermined action data 262 associated with the particular known user, where each of the known users may have the same and/or different predetermined actions data based on the preferences of each of the known users. For example, a first known user may have data stored in the predetermined action data 262 that allows the device 200 to generate and transmit all alert data 266 to the first known user's mother, while all other known users may indicate that all predetermined action data 262 may only be transmitted to themselves. Furthermore, the triggered known user alerts may comprise an early warning alert, a warning alert, and an emergency alert. Likewise, the triggered caregiver alerts may comprise a caregiver early warning alert, a caregiver warning alert, and a caregiver emergency alert.

In many embodiments, the vitals baseline data 263 may be any data related to one or more vital signs being monitored for each of the known users by the vitals monitoring logic 221 of FIG. 2A. The vitals baseline data 263 may include one or more vital signs for each of the known users including, but not limited to, body temperature, heart rate, oxygenation saturation (SpO2), blood-pressure, vocal features, and so on. Some of these vitals signs may be monitored through continuous tracking of basic vital signs such as blood pressure, heart rate, body temperature, and SpO2. For example, any one or more sensors described herein may be used and have the capability to continuously measure some, if not, all of these vital signs. In certain embodiments, the vitals baseline data 263 may comprise any data monitored, generated, and/or received from one or more of the personal computing devices 101-109 depicted in FIG. 1, such as the wearable computing device 106 and/or the mobile computing device 104 depicted in FIG. 1. For example, as described above, the wearable computing device 106 may be a smartwatch, but may also be used to track vital signs such as heart-rate, blood-pressure, and/or other desired vital sign data available from the computing device operating system.

As described above, the vitals baseline data 263 may be generated by each of the known users enrolling in a voice-based health detection system (or the like) and respectively determining each of the known users' baseline data for each of the vital signs being tracked and selected by the respective known user. The vitals baseline data 263 may be used in combination with other data types within the known user data 250 and with the threshold data 243 and captured data 241, such that the combination of this data and the one or more logics of the voice-based health detector application 220 may then continuously monitor all predetermined vital signs and provide feedback or alert data 266 to the known user once a combination of parameters have exceeded their predetermined thresholds. For example, the vitals baseline data 263 may also include any data related to any of the predetermined thresholds such as the dynamic thresholds, the static thresholds, and so on. In addition, the vitals baseline data 263 may include any data related to any vocal features extracted (or derived) from analyzing any of the captured data 241, signal data 244, voice data 261, and keyword data 267 depicted in FIGS. 2A-2B.

For example, the vitals baseline data 263 may include any vocal features extracted by the vocal features logic 225 of FIG. 2A from audio signals captured by vitals monitoring logic 221 of FIG. 2A, where each vocal feature stored in the vitals baseline data 263 corresponds to one or more particular monitored vital signs of the known user. The extracted vocal features may comprise vocal characteristics of the known user such as, but not limited to, vocal pitch, speed, range, weight, and timbre. In some embodiments, the device 200 of FIG. 2A may be configured to detect whether the extracted vocal features exceed their respective predetermined thresholds, where data of such vocal features and thresholds may be stored within the vitals baseline data 263.

In a number of embodiments, the personal information data 264 may further comprise the keyword data 267. The personal information data 264 may comprise any supplemental personal data that may be generated and associated with each of the known users. In some embodiments, the personal information data 263 may comprise relevant personal account and contact data such as names, addresses, telephone numbers, age, external factor metadata, associated personal computing devices, etc. For example, some or all personal account data may be any data associated with the known user that may be utilized to gain insight into the captured voice data 261, vitals baseline data 263, known user data 250, and/or any captured data 241 within the data store 240 of FIG. 2A. For example, user data may indicate that a user has their birthday and may then be utilized to further gain understanding (or at least generate an additional data point) when processing their voice data 261 and other subsequent data. The external factor metadata may include any additional data relating to the event and physical location where the data was captured. Some external factor metadata examples may be captured with the heuristic logic 229 and/or the like, where some of the examples may include the global positioning system (GPS) coordinates of where the known user lives, where the captured vital sign measurements were taken (e.g., during an outdoor activity, on vacation, at work, etc.), the time at which it was taken (e.g., late at night or first thing in the morning, etc.), what was the quality of the recording, how long the recording was, and so on.

Additionally, the keyword data 267 stored within the personal information data 264 may be personalized for each of the known users. For example, the keyword data 267 may include any data related to words, phrases, conversations, and/or the like that are associated with a particularly known user. For example, the voice-based health detection device 200 of FIG. 2A may be configured as a keyword spotter. The features extracted from the decimated audio samples are one or more signals in a time domain, a frequency domain, or both the time and frequency domains characteristic of keywords and vocal features may be trained to be recognized by one or more neural networks of the voice-based health detection device 200. The keyword data 267 may include any data related to any user-specified keywords that may be identified from any type of signals that the particular user wants to detect. For example, the user-specified keyword data may be spoken keywords, particular vocal features of the spoken keywords, non-verbal acoustic signals such as specific sounds, signals, and so on. In such example, the particular user may have generated and stored the user-specified keyword data in the keyword data 267, such that the voice-based health detection device 200 may recognize personalized words, phrases, and so on such as “Hi,” “Good morning,” “On,” “Off,” “Hotter”, and “Colder,” in addition to other, standard keywords that are already included and stored in the signal data 244 depicted in FIG. 2A.

In some embodiments, the medical history data 265 may comprise any data related to any medical information and detected medical data points that are private and associated with each of the respective known users. The medical history data 265 may include any personal and/or private information that may be particular to the known user such as prior medical events such as surgeries, speech impairments, etc., present medication being taken by the particularly known user, and so on. In some embodiments, one or more data points from the medical history data 265 may be used during the vitals processing logic 226 determination of the extracted vocal features against their predetermined thresholds, such as if the particular known user already has an existing speech impairment that needs to be taken into account and so on. Additionally, as described above, the medical history data 265 may be stored on the voice-based health detection device 200 unlike the data stores 140, 142 depicted in FIG. 1, where the medical history data 265 may be stripped of any particular private data that may not be transmitted and/or may be transmitted via the privacy logic 228 depicted in FIG. 2A. For example, the medical history data 265 may be transmitted using blockchain-based data transmission, hash-based data transmission, encryption-based data transmission, and/or any other similar protected data transmission.

In many embodiments, the alert data 266 may comprise any data associated with one or more generated and transmitted alerts for each of the known users. For example, the alert data 266 may include any known users alerts that were generated and transmitted in response to determining that the extracted features exceeded their respective predetermined thresholds. In some embodiments, the stored alert data 266 may be generated with the alert logic 227 of FIG. 2A configured to generate alert data that may trigger at least one or more of known user alerts and caregiver alerts in response to any of the triggered predetermined action data 262. The known user alerts may comprise an early warning alert, a warning alert, and an emergency alert. The caregiver alerts may comprise a caregiver early warning alert, a caregiver warning alert, and a caregiver emergency alert. In many embodiments, the known user and caregiver alerts stored in the alert data 266 may comprise any type of alert notification that may be used as an early health detection of a potential severe health issue associated with the particular known user.

It will be understood by those skilled in the art that the known user data 250 depicted herein with respect to FIGS. 2A-2B is only a single representation of potential known user data. For example, various embodiments may have known user data 250 pooled together such that all voice data 261 is stored together, all predetermined action data 262 for all known user entries is stored together, etc. Furthermore, other methods of storing known user data 250 may be utilized without limitation, such that the known user data 250 may be stored externally while other aspects are stored locally. For example, the known user data 250 may store the voice data 261 externally, while the other data types 262-266 may be stored locally to avoid exposing private data such as medical history data 265 and personal information data 264.

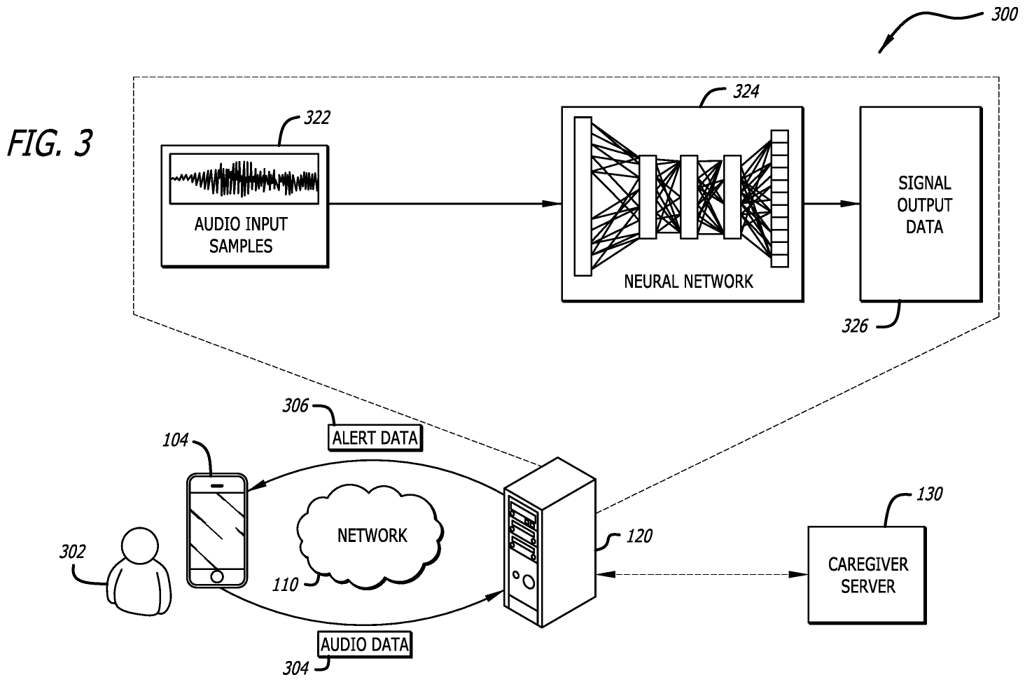

Referring now to FIG. 3, a detailed block diagram illustration of a voice-based health detection server 120 utilized in a voice-based health detection system 300 is shown, in accordance with embodiments of the disclosure. The voice-based health detection system 300 depicted in FIG. 3 may be similar to the voice-based health detection system 100 depicted in FIG. 1. In addition, the voice-based health detection server 120 depicted in FIG. 3 may be substantially similar to the voice-based health detection server 120 depicted in FIG. 1.

The voice-based health detection system 300 depicts an exemplary system for speech recognition and vocal features detection using the voice-based health detection server 120. As shown in FIG. 3, the voice-based health detection server 120 may, in many embodiments, be configured to provide audio input samples 322 to one or more neural networks 324, which may respectively process the provided audio input samples 322 to generate the signal output data 326. The design and utilization of the neural networks in this manner is described in greater detail within co-pending U.S. patent application Ser. No. 16/701,860, filed Dec. 3, 2019, which is assigned to the common assignee, the disclosure of which is incorporated herein by reference in its entirety.

In some embodiments, the voice-based health detection system 300 may comprise a user 302, a mobile computing device 104, a network 110, the voice-based health detection server 120, and a caregiver server 130. The mobile computing device 104, network 110, voice-based health detection server 120, and caregiver server 130 in FIG. 3 may be substantially similar to the mobile computing device 104, network 110, voice-based health detection server 120, and caregiver server 130 depicted in FIG. 1. In some embodiments, the user 302 may be a known user who uses the mobile computing device 104, where the mobile computing device 104 may be any type of computing devices described herein.

In many embodiments, the voice-based health detection system 300 may use the voice-based health detection server 120 to receive audio data 304 captured by the mobile computing device 104. The voice-based health detection server 120 may be configured to process the audio data 304 to detect (or identify, generate, etc.) one or more audio input samples 322 that are provided to a neural network 324, such as a digital neural network or the like. Additionally, the voice-based health detection server 120 may be configured to use signal output data 326 that may be generated by the neural network 324. The voice-based health detection server 120 may also be configured to generate alert data 306 based on the generated signal output data 326 if needed in response to the received audio data 304. Additionally, the voice-based health detection server 120 may be used to transmit data related to the generated alert data 306 to the caregiver server 130.

In some implementations, the voice-based health detection server 120 may receive a set of audio input samples 322. The server may receive data indicative of a time-frequency representation based on a set of audio input samples 322. The computing system 320 may provide, as input to a neural network, the time-frequency representation based on a set of audio input/waveform samples. The computing system 320 may identify one or more keywords spoken by the user 302 and may provide the identified keywords as the audio input samples 322 to the neural network 324.

In the illustrated example, the user 302 of the mobile computing device 104 may speak words and the mobile computing device 104 may respective record multi-channel audio that includes the speech (i.e., the spoken words). The mobile computing device 104 may transmit the recorded audio data signal 312 to the voice-based health detection server 120 over the network 110. The voice-based health detection server 120 may receive the audio data 304 to obtain the one or more audio input samples 322. For example, the voice-based health detection server 120 may identify a set of audio input (or waveform) samples 322 from the audio data 304 that may occur within a time window of audio data signal 304. The voice-based health detection server 120 may provide the audio waveform samples 322 to the neural network 324.

The neural network 324 may be configured and trained to act as an acoustic model. For example, the neural network 324 may indicate one or more likelihoods that may be implemented as time-frequency feature representations corresponding to different speech units, where the time-frequency feature representations may be output based on the audio input samples 322. In some embodiments, the neural network 324 may be configured to identify keywords from the received audio data and extract vocal features from the identified keywords. The extracted vocal features may comprise vocal characteristics of the user, such as vocal pitch, speed, range, weight, and timbre. The neural network 324 may also be configured to detect whether the extracted vocal features exceed a predetermined threshold and provide the signal output data 326 with the detected extracted features that have exceeded their predetermined threshold. The voice-based health detection server 120 may therefore use the provided signal output data 326 from the neural network 324 to provide early warning alert data 306 of potential health issues to the mobile computing device 104 of the user 302 in response to the detected vocal features exceeding the predetermined threshold.

Referring now to FIG. 4, an exemplary flowchart of a voice-based health detection process 400 for generating known user data is shown, in accordance with embodiments of the disclosure. The process 400 may be depicted as a flowchart used to personalize and update a voice-based health detection system by generating known user data that may be made available to a known user for the purpose of executing desired user-specific functions. The process 400 may be implemented with one or more computing devices and/or systems including, but not limited to, the voice-based health detection system 100 depicted in FIG. 1, the voice-based health detection device 200 depicted in FIG. 2A, the voice-based health detection system 300 depicted in FIG. 3, and/or the voice-based health detection server 120 depicted in FIGS. 1 and 3. Additionally, as described above in various embodiments, the process 400 may be implemented by way of one or more web-based applications and/or any other suitable software applications. In some embodiments, the application(s) may be implemented as a cloud-based application and/or distributed as a stand-alone software application, as desired, without limitations.

At block 410, the process 400 may begin with entering user-specified keyword data of a user. The entered user-specified keyword data enables the user, or a customer, to enter any desired target signals into the application. User-specified keyword data may be any type of signals (or target signals) that the user wants to detect. For example, the user-specified keyword data may be spoken keywords, non-verbal acoustic signals such as specific sounds, image types, and so on to be captured by one or more sensors such as any of the personal computing devices 101-109 depicted in FIG. 1 and/or the like. In an exemplary embodiment, the user may enter the desired keywords, and the sensors may recognize the personalized keywords, such as, by way of non-limiting example, “On,” “Off,” “Hotter”, and “Colder,” in addition to any other standard keywords that are already included in a keyword data store.

At block 420, the process 400 may generate vitals baseline data for the user-specified keyword data. The generated vitals baseline data may include one or more vocal features and baseline data related to the user-specified keywords. As described herein, once the desired keywords have been entered, the user may also establish the one or more vocal features and the baseline data based on the entered keywords. For example, a voice-based health detection device may be used to continuously monitor and detect temporal changes over time of the spoken keywords by the user. This allows the device to extract one or more predetermined vocal features of the spoken keywords, for example, to establish and determine trends and threshold measured readings of the extracted features over time. The extracted vocal features may include one or more vocal characteristics extracted from audio data (or audio signal, sample, etc.) that are particular to the user, including, but not limited to, vocal pitch, vocal speed, vocal range, vocal weight, vocal timbre, and so on. As such, any pre-determined vocal changes to the extracted features based on the vitals baseline data may be used to provide early health alert signals to the user and/or a caregiver of the user.

For example, the process 400 may allow the user to enroll in the above-referenced application and establish the respective features and the vitals baseline data in relation to the entered keywords. In many embodiments, the extracted features may be checked (or verified) against the generated baseline data. In many embodiments, the extracted vocal features may then be analyzed to determine whether any of the extracted vocal features have exceeded one or more predetermined thresholds. As described above, the vitals baseline data may include data of all the extracted vocal features detected over time, which is used to establish trends and threshold data of the extracted vocal features to help track one or more vital signs of the user. In many embodiments, the user's vitals baseline data may include one or more baseline thresholds associated with the extracted features which correspond to the monitored vital signs. For example, a first predetermined threshold may include a range of minimum and maximum data values for a vital sign being monitored for a user, where a particular data value may be generated for an extracted vocal feature of the user and typically falls within that threshold when the user is healthy.

Conversely, in other embodiments when the one or more extracted vocal features have exceeded their respective predetermined thresholds, one or more early sign health alert data signals may be generated and transmitted to the user, and/the extracted vocal features may be further processed to determine one or more subsequent actions (e.g., generate and transmit the early sign health alert signal to a primary caregiver of the user). As such, the early sign health alert data signal may provide the user with an early warning of a potential severe health issue and to seek further diagnosis of the potential severe health issue, where the potential severe health issue corresponds to one or more of the vital signs that are being monitored for the user based on the generated vitals baseline data.

At block 430, the process 400 may retrieve signal data from a data store associated with the user. The signal data may be comprised of standard keywords or the like that may be detected. At block 440, the process 400 may build a modified data store based on the combination of user-specified keyword data and the signal data. In some embodiments, the user-specified target signals may be labeled with suitable, corresponding labels, while all other signals may be identified by way of a generic label, such as “Other,” for example. Thereafter, the modified data store may then be used to train a neural network implementation. At block 450, the process 400 may train a neural network based on the modified data store to recognize both the combination of the user-specified keyword data and the signal data in the modified (or updated) data store. It is contemplated that the neural network implementation may be a software model of a neural network such as the neural network 324 depicted in FIG. 3. At block 460, the process 400 may generate known user data to train the neural network implementation. For example, the generated known user data may be used by a voice-based health detection device to detect the user-specified keywords in the modified data store.

At block 470, the process 400 may optionally translate the generated known user data into a file format suitable for being stored in a memory storage of a personal computing or other device. For example, the memory storage may be similar to the data store 240 depicted in FIG. 2A. In some embodiments, a programming file comprised of the generated known user data may be provided to an end-user upon purchasing the device. In some embodiments, the file may be programmed into one or more integrated circuits and/or logics that may be purchased by the end-user. Upon the end-user installing the file comprising the generated known user data into the device, either by way of the above-mentioned programming file, circuits, and/or logics, the device may detect the entered user-specified keywords in the modified data store. As will be appreciated by those skilled in the art, the training or implementation of the neural network is performed externally of the device and the resultant known user data is stored in the memory storage, the device may continue to monitor audio signals in the offline state (i.e., in absence of a cloud or other network connection).

Referring now to FIG. 5, an exemplary flowchart of a voice-based health detection process 500 is shown, in accordance with embodiments of the disclosure. The process 500 may be depicted as a flowchart used to generate early detection alert data for health issues of known users. The process 500 may be implemented with one or more computing devices and/or systems including, but not limited to, the voice-based health detection system 100 depicted in FIG. 1, the voice-based health detection device 200 depicted in FIG. 2A, the voice-based health detection system 300 depicted in FIG. 3, and/or the voice-based health detection server 120 depicted in FIGS. 1 and 3. Additionally, as described above in various embodiments, the process 500 may be implemented by way of one or more web-based applications and/or any other suitable software applications. In some embodiments, the application(s) may be implemented as a cloud-based application and/or distributed as a stand-alone software application, as desired, without limitations.

At block 510, the process 500 may receive audio signal data. For example, the received audio signal data may be provided in the form of raw analog audio signals, digital signal data and patterns that represent particular sounds or the like, and/or any other recognizable signal input, which are captured by one or more sensors such as any of the personal computing devices 101-109 depicted in FIG. 1. The received audio signal data may be captured from within a voice-based health detection device or may be remotely captured and transmitted to the voice-based health detection device for processing. At block 520, the process 500 may identify (or detect) one or more keywords within the received audio signal data. For example, the voice-based health detection device may detect the predetermined keywords within the received audio signal data. In many embodiments, the identified keywords are received from sounds, voices, or the like picked-up within a proximity of the device.

At block 530, the process 500 may extract vocal features from the identified keywords. For example, the one or more extracted vocal features are then processed and verified against the vitals baseline data associated with the known user. The extracted vocal features may particularly correspond to the identified keywords. The extracted vocal features may include one or more vocal characteristics generated by the known user and extracted from the identified keywords, which includes, but is not limited to, vocal pitch, vocal speed, vocal range, vocal weight, vocal timbre, and/or the like. At block 540, the process 500 may process one or more changes to the extracted vocal features. For example, the processed vocal features may be evaluated against the known user data associated with a specific known user. In addition, the processed vocal features may also be evaluated against the vitals baseline data associated with the known user. By completing this evaluation, the processed vocal features of the known user may allow for the identification of one or more changes that may be particular to the known user. This evaluation against a baseline specific to the known user can account for preexisting features such as, but not limited to, a speech impairment, local speech dialects, and/or accents.

At block 550, the process 500 may determine whether the extracted feature(s) have exceeded a predetermined threshold. If the extracted vocal features have not exceeded their respective predetermined thresholds, the process 500 may proceed back to block 510. For example, the extracted features are then analyzed to determine whether any of the extracted vocal features have exceeded their respective predetermined thresholds, while also considering one or more external factors that may impact the predetermined thresholds and/or processed vocal features (e.g. the heuristic logic 229 depicted in FIG. 2A may be utilized when considering such external factors). Additionally, as described above, the predetermined threshold may comprise at least one or more dynamic/static predetermined thresholds. For example, the predetermined threshold may comprise a static predetermined threshold that may be generated based on the vitals baseline data of the known user. In some embodiments, the static predetermined threshold may comprise a static range of minimum and maximum data values associated with the vitals baseline data of the known user. That is, the static predetermined threshold may have one minimum data value and one maximum data value for one vital sign, where both the minimum and maximum data values are fixed for the one vital sign, may not be changed, and/or do not otherwise take other external factors into consideration.

In additional embodiments, the predetermined threshold may comprise a dynamic threshold. The dynamic threshold may be generated based on a variety of changing factors including, but not limited to, the vitals baseline data of the known user. Moreover, the dynamic threshold may comprise a dynamic range of minimum and maximum data values associated with the vitals baseline data of the known user. For example, the dynamic threshold may be generated in conjunction with a vitals processing logic (e.g., the vitals processing logic 226 of FIG. 2A) that may be configured to dynamically adjust the dynamic range of minimum and maximum data values based on one or more external factors captured by a heuristic logic (e.g., the heuristic logic 229 of FIG. 2A). In some embodiments, the one or more captured external factors may comprise at least one or more of geographic location, time, date, real-time activities, physical location, ambient temperature, and/or monitored temperature. For example, on a hot day, the dynamic predetermined threshold may dynamically adjust the respective minimum and maximum data values for the monitored vital signs associated with the temperature of the known user. It is contemplated that a variety of dynamic thresholds can be configured based on any number of external factors.

Accordingly, at block 560 when the extracted vocal features have exceeded their respective thresholds, the process 500 may generate alert data in response to the extracted vocal features having exceeded their predetermined thresholds. For example, the generated alert data may be configured as a data command, a function call, a related predetermined action, a change in voltage within the device, and/or the like. At block 570, the process 500 may transmit the generated alert data to one or more computing devices. In addition, the transmitted alert data may trigger, in response to the generated alert data, predetermined action data associated with the known user, which may trigger at least one or more known user alerts and/or caregiver alerts. For example, the known user alerts may comprise an early warning alert, a warning alert, and an emergency alert, while the caregiver alerts may comprise a caregiver early warning alert, a caregiver warning alert, and a caregiver emergency alert. Lastly, the process 500 may be configured to, in response to the generated and transmitted alert data, transmit one of the known user alerts to a personal computing device of the known user; and/or one of the caregiver alerts to a caregiver server associated with the known user, where both known user and caregiver alerts comprise an alert notification of an early health detection of a potential severe health issue that is associated with the known user.

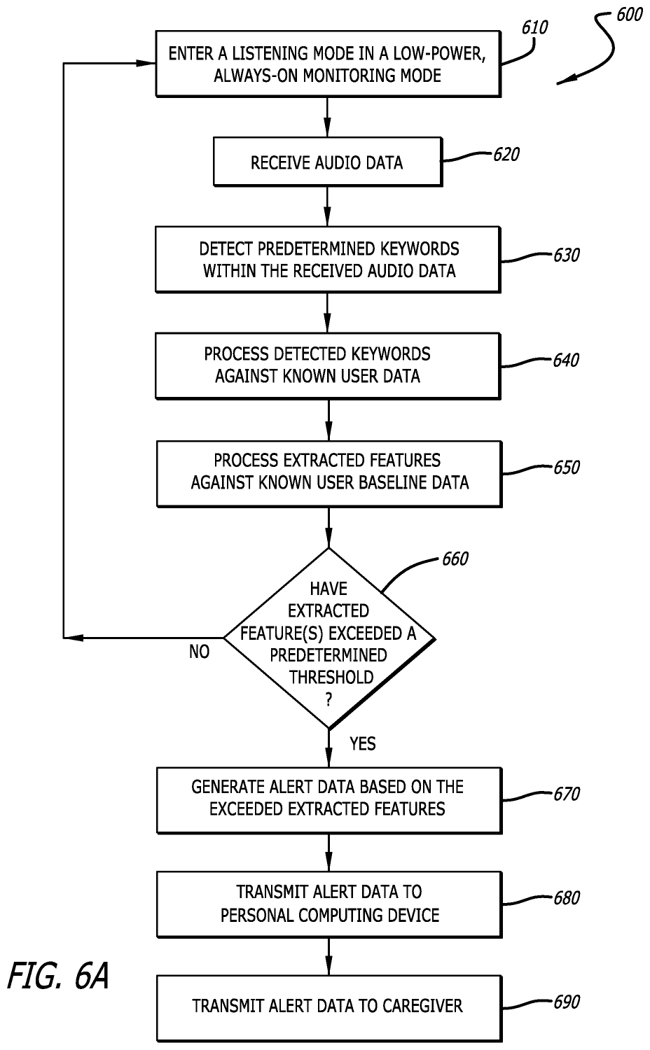

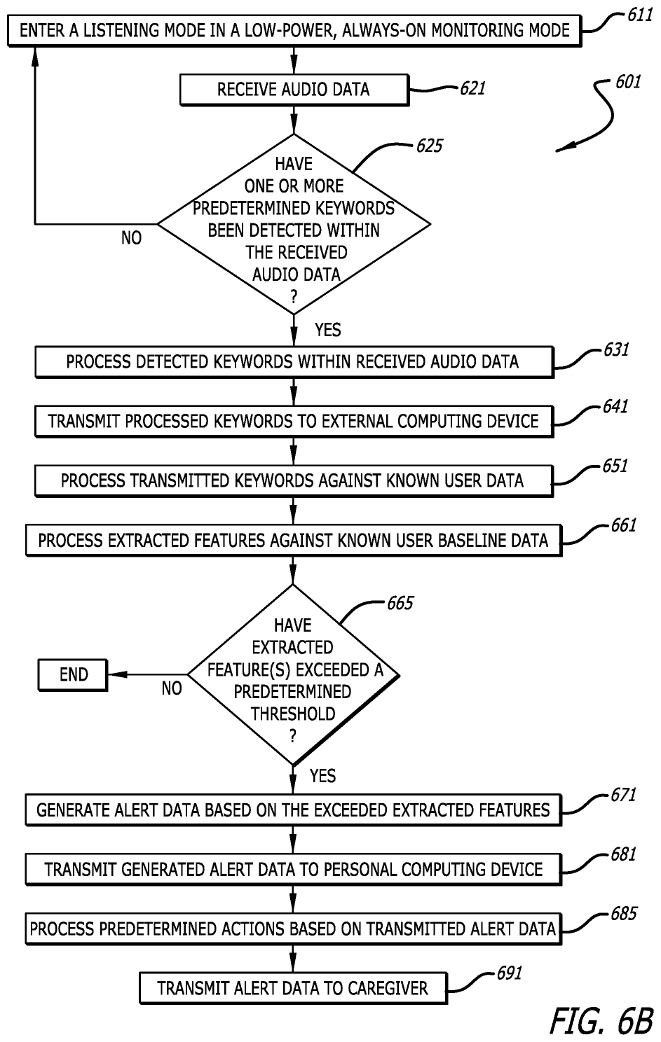

Referring now to FIG. 6A, an exemplary flowchart of an always-on voice-based health detection process 600 is shown, in accordance with embodiments of the disclosure. The process 600 may be depicted as a flowchart used to monitor on-device data of known users and generate early detection alert data for health issues of such known users. The process 600 may be implemented with one or more computing devices and/or systems including, but not limited to, the voice-based health detection system 100 depicted in FIG. 1, the voice-based health detection device 200 depicted in FIG. 2A, the voice-based health detection system 300 depicted in FIG. 3, and/or the voice-based health detection server 120 depicted in FIGS. 1 and 3. Additionally, as described above in various embodiments, the process 600 may be implemented by way of one or more web-based applications and/or any other suitable software applications. In some embodiments, the application(s) may be implemented in part or entirely as a cloud-based application and/or distributed as a stand-alone software application, as desired, without limitations.

At block 610, the process 600 may enter a listening mode in a low-power, always-on monitoring mode. In many embodiments, the process 600 may be implemented with a voice-based health detection device or the like. For example, the voice-based health detection device may be similar to the voice-based health detection device 200 depicted in FIG. 2A. In the following embodiments, the device may be used to enter into the listening mode which may be utilized by a sensor (or the like) and resides within the device in a low-power, always-on mode (or power consumption state) so that the sensor may provide low-latency recognition of any type of audio data signals.

At block 620, the process 600 may receive audio data from one or more sensors and/or personal computing devices of a user. For example, the device may receive the one or more audio signal inputs in the form of raw analog audio signals, digital signal data and patterns that represent particular sounds or the like, and/or any other recognizable signal input, which are captured from one or more audio data sources, sensors, and/or the like. The received audio signals may be captured from within the computing device or may be remotely captured and transmitted to the device for processing. At block 630, the process 600 may detect predetermined keywords within the received audio data. For example, the device may detect one or more user-specified keywords within the received audio data. At block 640, the process 600 may process the detected keywords against known user data associated with the user. For example, the transmitted keywords may be processed against the known user data similar to the known user data 250 depicted in FIGS. 2A-2B. For example, in some embodiments, the one or more detected keywords are then processed and checked against the known user data of one or more known users. The known user data may comprise data associated with the one or more known users who have been preauthorized to use the device. In addition to preauthorized known users, the known user data may also include keywords, features, and/or any other desired data based on the known users, such data may be depicted with the one or more data types in the known user data 250 depicted in FIG. 2B. In some embodiments, the known users may be determined by processing the detected keywords as a separate recognition network may be used to determine the sources of the known users. For example, the checking of the detected keywords against the known users may be done sequentially or in parallel with step 630.

At block 650, the process 600 may process extracted vocal features against known user baseline data. For example, the one or more extracted features may then be processed and verified against the vital baseline data in the known user data. As described herein, the extracted vocal features and the known user vital baseline data may correspond particularly to the detected keywords. At block 660, the process 600 may determine whether the processed and extracted vocal features have exceeded a predetermined threshold. For example, this determination may be similar to the determination at block 550 depicted above in FIG. 5. At block 670, the process 600 may generate alert data in response to the extracted vocal features that have exceeded their predetermined thresholds. At block 680, the process 600 may transmit the generated alert data to one or more personal computing devices. For example, the transmitted alert data may be transmitted in a recognizable form that may be received by any of the personal computing devices 101-109 of FIG. 1. At block 690, the process 600 may optionally transmit the generated alert data to a caregiver device, server, and/or system. For example, the transmitted alert data may be transmitted in a recognizable form that may be received by the caregiver server 130 of FIG. 1. The transmitted alert data received by the caregiver may alert them of an early health detection of a potential severe health issue associated with the user.

Referring now to FIG. 6B, an exemplary flowchart of an always-on voice-based health detection process 601 utilizing an external computing device is shown, in accordance with embodiments of the disclosure. The process 601 may be depicted as a flowchart used to receive off-device data of known users and generate early detection alert data for health issues of such known users. The process 601 depicted in FIG. 6B may be similar to the process 600 depicted in FIG. 6A with the exception that each (and/or most) of the depicted steps of the process 601 may be implemented in a cloud-based server and/or the like. The process 601 may be implemented with one or more computing devices and/or systems including, but not limited to, the voice-based health detection system 100 depicted in FIG. 1, the voice-based health detection device 200 depicted in FIG. 2A, the voice-based health detection system 300 depicted in FIG. 3, and/or the voice-based health detection server 120 depicted in FIGS. 1 and 3. Additionally, as described above in various embodiments, the process 601 may be implemented by way of one or more web-based applications and/or any other suitable software applications. In some embodiments, the application(s) may be implemented in part or entirely as a cloud-based application and/or distributed as a stand-alone software application, as desired, without limitations.

At block 611, the process 601 may enter a listening mode in a low-power, always-on monitoring mode. For example, this entered listening mode may be similar to the entered listening mode at block 610 depicted above in FIG. 6A. At block 621, the process 601 may receive audio data transmitted from one or more sensors and/or personal computing devices that may be located on a user. At block 625, the process 601 may then determine whether predetermined keywords have been detected within the received audio data. If no predetermined keywords are detected, the process 601 may proceed back to block 611. Conversely, if predetermined keywords are detected, the process 601 may proceed to block 631 and may process the detected keywords within received audio data. It should be understood that the previous blocks may be similar to the respective blocks depicted above in FIG. 6A.

At block 641, the process 601 may transmit the processed keywords to one or more external computing devices. As described above, the external computing devices may be implemented as a cloud-based device, server, system, and/or the like. For example, the cloud-based external computing devices may be configured to receive detected keywords from the one sensors and/or personal computing devices. At block 651, the process 601 may process the transmitted keywords against known user data. For example, the transmitted keywords may be processed against the known user data similar to the known user data 250 depicted in FIGS. 2A-2B. At block 661, the process 601 may process the extracted features against known user baseline data. At block 665, the process 601 may determine whether extracted features have exceeded a predetermined threshold. If the extracted vocal features have not exceeded their respective predetermined thresholds, the process 601 may proceed to end the process. Conversely, if the extracted vocal features have exceeded their respective predetermined thresholds, the process 601 may proceed to block 671 and may generate alert data based on the exceeded extracted features. It should be understood that the previous blocks may be similar to the respective blocks depicted above in FIG. 6A.

At block 681, the process 601 may transmit generated alert data to personal computing device. For example, the cloud-based device (or service, application, etc.) may transmit the generated alert data to any personal computing device associated with the user. At block 685, the process 601 may process predetermined actions based on transmitted alert data. For example, the processed predetermined actions may include generating alert notifications and/or triggering other desired actions associated with the user. At block 691, the process 601 may transmit the processed alert data to a caregiver. It should be understood that this block may be similar to the respective block(s) depicted above in FIG. 6A.

Information as shown and described in detail herein is fully capable of attaining the above-described objective(s) of the present disclosure, the presently preferred embodiment of the present disclosure, and is, thus, representative of the subject matter that is broadly contemplated by the present disclosure. The scope of the present disclosure fully encompasses other embodiments that might become obvious to those skilled in the art, and is to be limited, accordingly, by nothing other than the appended claims. Any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims.

Moreover, no requirement exists for a system or method to address each and every problem sought to be resolved by the present disclosure, for solutions to such problems to be encompassed by the present claims. Furthermore, no element, component, or method step in the present disclosure is intended to be dedicated to the public regardless of whether the element, component, or method step is explicitly recited in the claims. Various changes and modifications in form, material, work-piece, and fabrication material detail may be made, without departing from the spirit and scope of the present disclosure, as set forth in the appended claims, as might be apparent to those of ordinary skill in the art, are also encompassed by the present disclosure

What is claimed is:

1. A system for generating early detection data for health issues, comprising:
one or more sensors;
a processor communicatively coupled to the one or more sensors; and
a memory communicatively coupled to the processor, the memory comprising:
a vital monitoring logic to receive signal data from the one or more sensors;
a sensor output detector logic configured to identify characteristics from the received signal data;
a features logic configured to extract one or more features from the identified characteristics;
a vitals processing logic configured to determine whether the one or more extracted features exceed a predetermined threshold; and
an alert logic configured to generate alert data in response to the one or more extracted features exceeding the predetermined threshold.

2. The system of claim 1, wherein the one or more sensors are configured to monitor one or more vital signs from a known user.

3. The system of claim 1, wherein the signal data comprises audio data collected from an always-on device.

4. The system of claim 2, wherein the identified characteristics comprise at least one or more characteristics associated with the known user, wherein the sensor output detector logic is further configured to detect words associated with the known user, and wherein the identified characteristics include at least one or more of the detected words.

5. The system of claim 4, the memory further comprising:
a sample processing logic configured to:
generate characteristics data based on the identified characteristics from the sensor output detector logic; and
process the characteristics data against known user data associated with the known user.

6. The system of claim 5, wherein the one or more extracted features are extracted from the processed characteristics data of the known user.

7. The system of claim 6, wherein the one or more extracted features comprise at least one or more of a vocal pitch, a vocal speed, a vocal range, a vocal weight, and a vocal timbre.

8. The system of claim 2, wherein the one or more sensors comprise at least one or more of wearable devices, smart hearables, head mounted displays, gaming consoles, mobile computing devices, computing tablets, smart remote controls, voice-based speakers, and smart home devices.

9. The system of claim 7, wherein the determination of the extracted features further includes determining whether the extracted features exceed the predetermined threshold based on vitals baseline data of the known user.

10. The system of claim 1, wherein the system operates in a low-power, always-on mode such that the system remains continuously ready to receive the signal data.

11. The system of claim 2, wherein the alert logic is further configured to trigger, in response to the generated alert data, one or more actions associated with predetermined action data related to the known user.

12. The system of claim 11, wherein the alert logic is further configured to trigger at least one or more of known user alerts and caregiver alerts in response to the triggered predetermined action data of the known user.

13. The system of claim 12, wherein the known user alerts comprise an early warning alert, a warning alert, and an emergency alert.

14. The system of claim 13, where in the caregiver alerts comprise at least one of a caregiver early warning alert, a caregiver warning alert, and a caregiver emergency alert.

15. The system of claim 14, wherein, in response to the generated alert data, the alert logic is further configured to:
transmit one of the known user alerts to a personal computing device of the known user; and
transmit one of the caregiver alerts to a caregiver server associated with the known user, wherein both known user and caregiver alerts comprise an alert notification of an early health detection of a potential severe health issue that is associated with the known user.

16. The system of claim 15, wherein the memory further comprises a privacy logic configured to protect medical history data associated with the known user, and wherein the privacy logic is further configured to transmit the protected medical history data of the known user to the caregiver server, and to receive data in response to the transmitted medical history data from the caregiver server, via one or more forms of data transmission.

17. The system of claim 16, wherein the one or more forms of data transmission comprise one of: blockchain-based data transmission, hash-based data transmission, and encryption-based data transmission.

18. The system of claim 9, wherein the predetermined threshold comprises a static predetermined threshold, wherein the static predetermined threshold is generated based on the vitals baseline data of the known user, and wherein the static predetermined threshold comprises a static range of minimum and maximum data values associated with the vitals baseline data of the known user.

19. The system of claim 9, wherein the predetermined threshold comprises a dynamic predetermined threshold, wherein the dynamic predetermined threshold is generated based on the vitals baseline data of the known user, and wherein the dynamic predetermined threshold comprises a dynamic range of minimum and maximum data values associated with the vitals baseline data of the known user.

20. The system of claim 19, wherein the vitals processing logic is further configured to dynamically adjust the dynamic range of minimum and maximum data values of the dynamic predetermined threshold based on one or more external factors that are captured by a heuristic logic, and wherein the one or more captured external factors comprise at least one or more of geographic location, time, date, real-time activities, physical location, ambient temperature, and monitored temperature.

21. A method for detecting vocal changes to provide early detection data of severe health issues, comprising:
receiving signal data from one or more sensors associated with a known user;
identifying characteristics from the received signal data;
extracting one or more features from the identified characteristics;
processing the one or more extracted features against vitals baseline data from the known user;
determining whether the one or more extracted features exceed a predetermined threshold;
generating alert data based on the one or more extracted features that have exceeded the predetermined threshold; and
transmitting the generated alert data to a personal computing device of the known user.

22. The method of claim 21, further comprising:
generating characteristics data based on the identified characteristics; and
processing the characteristics data against known user data associated with the known user, wherein the identifying characteristics from the received signal data further comprises detecting words from the received signal data, and wherein the identified characteristics include at least one or more of the detected words.

23. The method of claim 22, wherein the one or more extracted features are extracted from the processed characteristics data of the known user, and wherein the one or more extracted features comprise at least one or more of a vocal pitch, a vocal speed, a vocal range, a vocal weight, and a vocal timbre.

24. The method of claim 21, wherein the one or more sensors comprise at least one or more of wearable devices, smart hearables, head mounted displays, gaming consoles, mobile computing devices, computing tablets, smart remote controls, voice-based speakers, and smart home devices.

25. The method of claim 23, wherein the determination of the extracted features further includes determining whether the extracted features exceed the predetermined threshold based on vitals baseline data of the known user.

26. A system for remotely generating early detection data for health issues, comprising:
a processor; and
a memory communicatively coupled to the processor, the memory comprising:
a sample processing logic configured to:
receive characteristics from signal data captured by one or more computing devices associated with a known user;
generate characteristics data based on the identified characteristics; and
process the generated characteristics data against known user data;
a features logic configured to extract one or more features from the processed characteristics data;
a vitals processing logic configured to:
process the one or more extracted features against known user baseline data; and
determine whether the one or more processed features exceed a predetermined threshold; and
an alert logic configured to:
generate alert data in response to the one or more extracted features exceeding the predetermined threshold; and
transmit the generated alert data to the one or more computing devices associated with the known user.

27. The system of claim 26, further comprising a privacy logic configured to receive and transmit any privacy data.

28. The system of claim 27, wherein the privacy data comprises medical history data, and wherein the privacy logic is further configured to transmit the privacy data related to the medical history data that is private and associated with the known user.
